(19) United States
(12) Patent Application Publication
(10) Pub. No.: US 2011/0165130 A1
(43) Pub. Date: Jul. 7, 2011

(54) Methods for Preparing Human Skin Substitutes from Human Pluripotent Stem Cells

(75) Inventor: Hind Guenou, Paris (FR)

(73) Assignee: INSERM

(21) Appl. No.: 12/999,129

(22) PCT Filed: Jun. 23, 2009
(86) PCT No.: PCT/EP09/57817

§ 371 Date: Mar. 4, 2011

Related U.S. Application Data
(60) Provisional application No. 61/090,957, filed on Aug. 22, 2008.

(30) Foreign Application Priority Data
Jun. 25, 2008 (EP) 08305320.7
Aug. 4, 2008 (EP) 08305447.8
Jan. 28, 2009 (EP) 09305077.1

(51) Int. Cl. A61K 35/36; C12Q 1/02; C12N 5/071; A61P 17/02

(52) U.S. Cl. 424/93.7; 435/29; 435/366

(57) ABSTRACT

The present invention relates to an ex vivo method for obtaining a population of human keratinocytes derived from human pluripotent stem cells comprising a step of co-culturing human pluripotent stem cells with cells that support ectodermal differentiation in presence of an agent that stimulates epidermal induction and a agent that stimulates terminal differentiation of keratinocytes. A further object of the invention relates to a method for preparing a human skin substitute comprising a step of providing an organotypic culture of the substantially pure homogenous population of human keratinocytes derived from human pluripotent stem cells obtained according to the method of the invention.

### FIELD OF THE INVENTION

The present invention relates to ex vivo methods for obtaining populations of human keratinocytes derived from human pluripotent stem cells and methods for preparing human skin substitutes.

### BACKGROUND OF THE INVENTION

The skin consists of self-renewing layers organized into functional units of differentiating cells with their origin in a single basal stratum of proliferating keratinocytes. The dead and dying cells that comprise the stratum corneum are continually shed during desquamation and replaced by cells derived from epidermal stem cells found in the stratum germinativum. Loss of epidermal function leads to loss of thermal regulation, reduced microbial defences, risks of desiccation, inhibited wound repair, and cosmetic concerns. In the absence of sufficient autologous donor for skin grafts, coverage of wounds with cultured human keratinocytes represents a promising option for treatment.

Furthermore, in vitro and in vivo models for human skin may represent tremendous tools for studying the lineage of epidermis cells or for testing cosmetic and pharmaceutical compounds for therapeutic or toxicological effects. For example the need for in vitro models is strengthened by the fact that there is an incentive to provide an alternative to the use of animals for testing compounds and formulations.

In addition, a number of diseases affect the function of keratinocytes, either cell autonomously or through alteration of their ability to form the pluristratified epidermal tissue. In vitro and in vivo models for human skin may represent ways to reveal molecular mechanisms of diseases and, as a consequence, identify pharmacological or biological compounds endowed with therapeutic potentials.

Thus, there is a need for methods for obtaining populations of human keratinocytes that may then be useful for skin therapy or for obtaining in vitro and in vivo models for human skin.

Embryonic stem cells and somatic cells that are genetically reprogrammed in order to replicate all characteristics of embryonic stem cells (such as, for example, those called “iPS” cells, for “induced pluripotent stem” cells) are pluripotent stem cells with an extensive proliferative capacity and accordingly offer a great potential use in research and medicine. Several attempts have therefore been described in the prior art for obtaining human keratinocytes from pluripotent stem cells. For example, document WO02/097068 describes a method for inducing keratinocyte differentiation of embryonic stem cells. Further studies report the use of embryonic stem cells for obtaining population of human keratinocytes (Coraux C. et al. 2003; Ji L. et al. 2006; Metallo C M. et al. 2007; and Aberdam E. et al. 2008). However, up to now, the methods of the prior art have failed to obtain human keratinocytes derived from human pluripotent stem cells that would demonstrate an ability to form a pluristratified epidermis (in vitro or following xenografting in animals), when treated according to techniques that were shown instrumental when using adult basal keratinocytes from donors (see, e.g., Green, 2008).

### SUMMARY OF THE INVENTION

The present invention relates to an ex vivo method for obtaining a population of human keratinocytes derived from human pluripotent stem cells comprising a step of co-culturing human pluripotent stem cells with cells that support ectodermal differentiation in presence of an agent that stimulates epidermal induction and an agent that stimulates terminal differentiation of keratinocytes.

The invention also relates to an ex vivo method for obtaining a population of human keratinocytes derived from human pluripotent stem cells, said method comprising a step of culturing human pluripotent stem cells on a cell culture surface coated with a layer of feeder fibroblasts in the presence of a keratinocyte culture medium supplemented with BMP-4 and ascorbic acid.

The present invention also relates to an isolated substantially pure homogenous population of human keratinocytes derived from human pluripotent stem cells obtainable by the method as above described.

The present invention also relates to pharmaceutical composition comprising a substantially pure homogenous population of human keratinocytes derived from human pluripotent stem cells of the invention and optionally a pharmaceutically acceptable carrier or excipient.

The present invention also relates to a method for preparing a human skin substitute comprising a step consisting of providing an organotypic culture of the substantially pure homogenous population of human keratinocytes derived from human pluripotent stem cells of the invention.

The invention also relates to a human skin substitute obtainable by the method as above described.

The invention also relates to a method for grafting an animal with a human skin substitute as described above.

The invention also relates to an animal model for human skin obtainable by the method as above described.

Finally, the invention relates to the human skin substitute as above described for the treatment of a pathology associated with skin damage.

### DETAILED DESCRIPTION OF THE INVENTION

### Definitions

As used herein, the term “marker” refers to a protein, glycoprotein, or other molecule expressed on the surface of a cell or into a cell, and which can be used to help identify the cell. A marker can generally be detected by conventional methods. Specific, non-limiting examples of methods that can be used for the detection of a cell surface marker are immunohistochemistry, fluorescence activated cell sorting (FACS), and enzymatic analysis.

The term “population of human keratinocytes” refers to a population of cells that is able to reconstruct the human epidermis and that is characterized by the capacity to produce keratin in the process of differentiating into the dead and fully keratinized cells of the stratum corneum. Markers of basal keratinocytes include markers of basal layer with keratin 5, 14 (K5/K14) and transcription factor p63, markers of supra basal layer with keratin 1 and keratin 10 (K1/K10), involucrin, fillagrin and markers specific of dermal-epidermal junction with integrins alpha6 and beta4, laminin-5 and collagen VII.

As used herein, the term “human pluripotent stem cell” refers to any human precursor cell that has the ability to form any adult cell.

As used herein, the term “human embryonic stem cells” or “hES cells” or “hESC” refers to human precursor cells that have the ability to form any adult cell. hES cells are derived from fertilized embryos that are less than one week old.

As used herein, the term “human induced pluripotent stem cells” or “human iPS cells” or “human iPSCs” refers to a type of human pluripotent stem cell artificially derived from a human non-pluripotent cell (e.g. an adult somatic cell). Human induced pluripotent stem cells are identical to human embryonic stem cells in the ability to form any adult cell, but are not derived from an embryo. Typically, a human induced pluripotent stem cell may be obtained through the induced expression of Oct3/4, Sox2, Klf4, and c-Myc genes in any adult somatic cell (e.g. fibroblast). For example, human induced pluripotent stem cells may be obtained according to the protocol as described by Takahashi K. et al. (2007), by Yu et al. (2007) or else by any other protocol in which one or the other agents used for reprogramming cells in these original protocols are replaced by any gene or protein acting on or transferred to the somatic cells at the origin of the iPS lines. Basically, adult somatic cells are transfected with viral vectors, such as retroviruses, which comprises Oct3/4, Sox2, Klf4, and c-Myc genes.

The term “substantially pure homogenous population”, as used herein, refers to a population of cells wherein the majority (e.g., at least about 80%, preferably at least about 90%, more preferably at least about 95%) of the total number of cells have the specified characteristics of the keratinocytes of interest.

As used herein, the term “isolated” refers to a cell or a population of cells which has been separated from at least some components of its natural environment.

As used herein, the term “keratinocyte culture medium” refers to a culture medium that contains nutrients necessary to support the growth, proliferation and survival of human keratinocytes. Thus, an appropriate culture medium according to the invention may consist in a minimal medium in which cells can grow, such as for example Dulbecco modified Eagle's minimal essential medium (DMEM), which is supplemented with at least 10% of fetal calf serum (FCS). In another particular embodiment the culture medium consists in a FAD medium composed of ⅔ DMEM, ⅓ HAM:F12 and 10% of fetal calf serum (FCII, Hyclone) supplemented with 5 μg/ml insulin, 0.5 μg/ml hydrocortisone, 10⁻¹⁰M cholera toxin, 1.37 ng/ml triodothyronin, 24 μg/ml adenine and 10 ng/ml recombinant human EGF.

The term “cell culture surface” or “cell culture matrix” refers to every type of surface or matrix suitable for cell culture. The term “cell culture surface” includes but is not limited to tissue culture plate, dish, well or bottle. In a particular embodiment, the culture surface is plastic surface of the culture plate, dish, well or bottle. The cell culture surface is to be compatible with the coating of dermis fibroblasts.

As used herein, the expression “cells that support ectodermal differentiation” refers to cells that provide an appropriate substrate and which secrete appropriate factors to support the growth and the differentiation of human pluripotent stem cells. In a particular embodiment, cells that support ectodermal differentiation are selected from the group of fibroblasts, more particularly of human fibroblasts and more particularly of dermis fibroblasts. In a particular embodiment, the cells that support ectodermal differentiation are mitomycin-inactivated human dermis fibroblasts.

As used herein, the expression “feeder fibroblasts” refers to cells that serve as a basal layer for pluripotent stem cells and provide secreted factors, extracellular matrix, and cellular contacts for the maintenance of stem cells in the undifferentiated state without losing pluripotency. Feeder cells can be inactivated by gamma irradiation or mitomycin. According to an embodiment of the invention, the feeder fibroblasts may be from the group of fibroblasts, more particularly of human fibroblasts and more particularly of dermis fibroblasts, including dermis fibroblast cell lines. Examples of dermis fibroblast cell lines include but are not limited to CCD-1112SK (Hovatta O, et al. 2003) and 3T3-J2 (Rheinwald J G et al. 1975). In a particular embodiment, dermis fibroblasts are previously treated to stop their proliferation before to be coated in the culture surface. Therefore, dermis fibroblasts may be irradiated or treated with a cell cycle blocking agent such as mitomycin.

As used herein, the term “dermis fibroblast” refers to a population of cells that synthesizes and maintains the extracellular matrix of dermis. Specific markers of dermis fibroblasts include vimentin and FAP (fibroblast activation protein).

As used herein, the expression “agent that stimulates epidermal induction” refers to an agent that is capable of inducing the expression of epidermal markers such as keratin 8, keratin 18, keratin 5 and keratin 14. Typically an agent that stimulates epidermal induction inhibits trophoblast and mesoderm induction.

In a particular embodiment, the agent that stimulates epidermal induction is selected from the group consisting of Bone Morphogenetic Proteins (such as BMP-2, BMP-4 and BMP-7), receptor-regulated Smad proteins (such as Smad 1, Smad 5 and Smad 9) and ligands of the TGF-beta family (such as Growth and Differentiation Factor 6 GFD-6) (Moreau et al., 2004). In a preferred embodiment the agent that stimulates epidermal induction is selected from the group consisting of BMP-, BMP-4, BMP-7, Smad1, Smad5, Smad7 and GFD-6. In a preferred embodiment, the agent that stimulates epidermal induction is BMP-4.

The term “BMP-4” refers to Bone morphogenetic protein 4. BMP-4 is a polypeptide belonging to the TGF-β superfamily of proteins. An exemplary native BMP-4 amino acid sequence is provided in GenPept database under accession number AAC72278.

As used herein, the expression “agent that stimulates terminal differentiation of keratinocytes” refers to an agent that stimulates the expression of keratin 5 and keratin 14. Indeed, keratin 5 and keratin 14 are markers of the basal keratinocytes which are capable of terminal differentiation in 3D culture. In one particular embodiment, the agent that stimulates terminal differentiation of keratinocytes is selected from the group consisting of ascorbic acid and retinoic acid.

The term “ascorbic acid” refers to (R)-3,4-dihydroxy-5-((S)-1,2-dihydroxyethyl)furan-2(5H)-one which has the formula of:

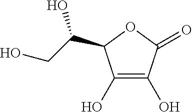

As used herein, the term “organotypic culture” refers to a three-dimensional tissue culture where cultured cells are used reconstruct a tissue or organ in vitro.

As used herein, the term “pathologies” refers to any disease or condition associated with skin damage. The term “pathology associated with skin damage” refers to any disease or clinical condition characterized by skin damage, injury, dysfunction, defect, or abnormality. Thus, the term encompasses, for example, injuries, degenerative diseases and genetic diseases. In certain embodiments, pathologies of interest are genodermatosis such as Epidemolysis bullosa, Xeroderma pigmentosum, ichthyosis, ectodermal dysplasia, kindler syndrome and others.

As used herein, the term “subject” refers to a mammal, preferably a human being, that can suffer from pathology associated with skin damage, but may or may not have the pathology.

In the context of the invention, the term “treating” or “treatment”, as used herein, refers to a method that is aimed at delaying or preventing the onset of a pathology, at reversing, alleviating, inhibiting, slowing down or stopping the progression, aggravation or deterioration of the symptoms of the pathology, at bringing about ameliorations of the symptoms of the pathology, and/or at curing the pathology.

### METHODS OF THE INVENTION

The present invention relates to an ex vivo method for obtaining a population of human keratinocytes derived from human pluripotent stem cells comprising a step of co-culturing human pluripotent stem cells with cells that support ectodermal differentiation in presence of an agent that stimulates epidermal induction and a agent that stimulates terminal differentiation of keratinocytes.

The human keratinocytes derived from human pluripotent stem cells obtainable by the method as above described are able to recapitulate all morphological and functional attributes of human basal keratinocytes. Indeed the inventors demonstrated that said cells are able to reconstruct a human epidermis (in vitro and in vivo) and that are characterized by the capacity to produce keratin. More particularly said cells express markers of basal keratinocytes that include markers of basal layer with keratin 5, 14 (K5/K14) and transcription factor p63, markers of supra basal layer with keratin 1 and keratin 10 (K1/K10), involucrin, fillagrin and markers specific of dermal-epidermal junction with integrins alpha6 and beta4, laminin-5 and collagen VII. They may also express keratin 19, which is a marker of skin stem cells, as well as keratin 3 and 12, which are markers of the corneal cells.

An embodiment of the invention relates to an ex vivo method for obtaining a population of human keratinocytes derived from human pluripotent stem cells, said method comprising a step of culturing human pluripotent stem cells on a cell culture surface coated with a layer of feeder fibroblasts in the presence of a keratinocyte culture medium supplemented with BMP-4 and ascorbic acid.

In a particular embodiment, human pluripotent stem cells include but are not limited to embryonic stem cells (hES cells) or human induced pluripotent stem cells (human iPS cells).

According to an embodiment of the invention, hES cells may be selected from any hES cell lines. Examples of hES cell lines include but are not limited to, SA-01, VUB-01, H1 (Thomson J A et al 1998), and H9 (Amit M et al. 2000). According to the invention hES cells are not previously cultured in the presence of LIF as described in the international patent application WO2002/097068. Moreover, according to the invention it shall be understood that hES cells are not previously differentiated in embryoid bodies as described in Metallo C M. et al. (2007) or in Ji L; et al. (2006).

According to an embodiment of the invention human iPS cells may be selected from any human iPS cell lines. Examples of human iPS cell lines include but are not limited to clones 201B (Takahashi et al., 2007) and iPS (Foreskin or IMR90)-1-MCB-1 (Yu et al., 2007).

Alternatively, hES cells or human iPS cells may be selected from master cell banks that may be constituted in a therapeutic purpose. In a preferred manner, hES cells or human iPS may be selected to avoid or limit immune rejection in a large segment of the human population. Typically hES cells or human iPS cells are HLA-homozygous for genes encoding major histocompatibility antigens A, B and DR, meaning that they have a simple genetic profile in the HLA repertory. The cells could serve to create a stem cell bank as a renewable source of cells that may be suitable for preparing human skin substitutes for use in cell therapy of pathologies associated with skin damage (e.g. wound, burns, irradiation, disease-related abnormalities of epidermis . . . ).

In another particular embodiment, human pluripotent stem cells may carry a mutation or a plurality of mutations that are causative for a genetic disease of the human skin.

According to an embodiment of the invention, the cell culture surface is selected in the manner that dermis fibroblasts may naturally adhere on it. Various materials of cell culture surface may be selected. Examples of such materials include but are not limited to tissue culture dishes or dishes coated with gelatine.

In a particular embodiment, the keratinocyte culture medium may be supplemented with one or more agents selected from the group consisting of glutamine, epidermal growth factor (EGF), sodium pyruvate, adenine, insulin, hydrocortisone, choleric toxin and triodothyronin. In a particular embodiment, the keratinocyte medium culture is the one described by Rheinwald J G. et al. (1975).

According to an embodiment of the invention, the concentration of ascorbic acid in the keratinocyte culture medium may vary from 0.01 mM to 1 mM. In a particular embodiment the concentration of ascorbic acid is 0.3 mM.

The concentration of BMP-4 in the keratinocyte culture medium may vary from 0.02 nM to 77 nM or 0.3 ng/ml to 1000 ng/ml. In a particular embodiment the concentration of BMP-4 is 0.5 nM.

According to the invention, human pluripotent stem cells (e.g. hES cells or human iPS cells) are cultivated for a time period sufficient for allowing the complete differentiation of said cells in a population of cells that recapitulate all morphological and functional attributes of human basal keratinocytes (“human keratinocytes derived from human pluripotent stem cells”). According to a particular embodiment, the time period may vary from 20 days to 60 days, preferably 20 days to 40 days.

A further object of the invention relates to an isolated population of human keratinocytes derived from human pluripotent stem cells obtainable by a method as above described.

According to another embodiment, the method of the invention may further comprise a step of culturing the population of human keratinocytes derived from human pluripotent stem cells obtained as previously described on a cell culture surface coated with a layer of dermis fibroblasts in the presence of a keratinocyte culture medium devoid of acid ascorbic and BMP-4. The further step may be suitable to obtain a substantially pure homogenous population of human keratinocytes derived from human pluripotent stem cells.

Dermis fibroblasts, cell culture surface and keratinocyte culture medium may be the same as previously described provided that the keratinocyte culture medium is not supplemented with acid ascorbic and BMP-4.

A further object of the invention relates to an isolated substantially pure homogenous population of human keratinocytes derived from human pluripotent stem cells obtainable by a method as above described.

### Pharmaceutical Compositions

The substantially pure homogenous population of human keratinocytes derived from human pluripotent stem cells obtained according to the method of the invention may be then suitable for skin therapy.

Therefore the invention relates to a pharmaceutical composition comprising a substantially pure homogenous population of human keratinocytes derived from human pluripotent stem cells of the invention and optionally a pharmaceutically acceptable carrier or excipient. In certain embodiments, a pharmaceutical composition may further comprise at least one biologically active substance or bioactive factor.

As used herein, the term “pharmaceutically acceptable carrier or excipient” refers to a carrier medium which does not interfere with the effectiveness of the biological activity of the progenitor cells, and which is not excessively toxic to the host at the concentrations at which it is administered. Examples of suitable pharmaceutically acceptable carriers or excipients include, but are not limited to, water, salt solution (e.g., Ringer's solution), oils, gelatines, carbohydrates (e.g., lactose, amylase or starch), fatty acid esters, hydroxymethylcellulose, and polyvinyl pyrroline. Pharmaceutical compositions may be formulated as liquids, semi-liquids (e.g., gels) or solids (e.g., matrix, lattices, scaffolds, and the like).

As used herein the term “biologically active substance or bioactive factor” refers to any molecule or compound whose presence in a pharmaceutical composition of the invention is beneficial to the subject receiving the composition. As will be acknowledged by one skilled in the art, biologically active substances or bioactive factors suitable for use in the practice of the present invention may be found in a wide variety of families of bioactive molecules and compounds. For example, a biologically active substance or bioactive factor useful in the context of the present invention may be selected from anti-inflammatory agents, anti-apoptotic agents, immunosuppressive or immunomodulatory agents, antioxidants, growth factors, and drugs.

A related aspect of the invention relates to a method for treating a subject suffering from a pathology associated with skin damage, said method comprising a step of administering to the subject an efficient amount of a substantially pure homogenous population of human keratinocytes derived from human pluripotent stem cells of the invention (or a pharmaceutical composition thereof).

As used herein, the term “efficient amount” refers to any amount of a substantially pure homogenous population of human keratinocytes derived from human pluripotent stem cells (or a pharmaceutical composition thereof) that is sufficient to achieve the intended purpose.

The substantially pure homogenous population of human keratinocytes derived from human pluripotent stem cells (or a pharmaceutical composition thereof) of the invention may be administered to a subject using any suitable method.

The substantially pure homogenous population of human keratinocytes derived from human pluripotent stem cells of the invention may be implanted alone or in combination with other cells, and/or in combination with other biologically active factors or reagents, and/or drugs. As will be appreciated by those skilled in the art, these other cells, biologically active factors, reagents, and drugs may be administered simultaneously or sequentially with the cells of the invention.

In certain embodiments, a treatment according to the present invention further comprises pharmacologically immunosuppressing the subject prior to initiating the cell-based treatment. Methods for the systemic or local immunosuppression of a subject are well known in the art.

Effective dosages and administration regimens can be readily determined by good medical practice based on the nature of the pathology of the subject, and will depend on a number of factors including, but not limited to, the extent of the symptoms of the pathology and extent of damage or degeneration of the tissue or organ of interest, and characteristics of the subject (e.g., age, body weight, gender, general health, and the like).

### Human Skin Substitutes and Animal Models of the Invention

The substantially pure homogenous population of human keratinocytes derived from human pluripotent stem cells of the invention may be also suitable for preparing human skin substitutes.

Typically the human skin substitute of the invention comprises a pluristratified epidermis which results from the in vitro derived culture of the substantially pure homogenous population of human keratinocytes derived from human pluripotent stem cells as above described that has stratified into squamous epithelia. In a particular embodiment, the human skin substitute of the invention may comprise a pluristratified epidermis as above described and a dermis.

Therefore a further aspect of the invention relates to a method of preparing a human skin substitute comprising a step consisting of providing an organotypic culture of the substantially pure homogenous population of human keratinocytes derived from human pluripotent stem cells of the invention.

Full stratification and histological differentiation of the substantially pure homogenous population of human keratinocytes derived from human pluripotent stem cells of the invention can be achieved by the use of three-dimensional organotypic culture methods (Doucet O, et al. 1998; Poumay y. et al. 2004; Gache Y. et al. 2004). For example, when in vitro cultures of the substantially pure homogenous population of human keratinocytes derived from human pluripotent stem cells of the invention are grown at an air-liquid interface, a highly ordered stratum corneum is formed.

In a particular embodiment, human skin substitutes according to the invention may be generated as described by Poumay, Y et al. 2004. Culture of the substantially pure homogenous population of human keratinocytes derived from human pluripotent stem cells of the invention may be performed on polycarbonate culture inserts. These cells may be maintained for 11 days in Epilife medium supplemented with 1.5 mM CaCl2 and 50 μg/ml ascorbic acid. The cells were exposed to the air-liquid interface by removing the culture medium for 10 days.

In a particular embodiment, the substantially pure homogenous population of human keratinocytes derived from human pluripotent stem cells is previously seeded on a cell culture matrix populated with human dermis fibroblasts before providing an organotypic culture of it as above described. This particular embodiment allows obtaining a human skin substitute which comprises dermis and epidermis. Such a method may be performed through the protocol as described by Del Rio M. et al. (2002) or Larcher F. et al. (2007). For example, the substantially pure homogenous population of human keratinocytes derived from human pluripotent stem cells of the invention may be seeded on a fibrin matrix populated with live dermis fibroblasts. Organotypic cultures are then grown submerged up to keratinocyte confluence, and finally maintained at the air-liquid interface for 7 days to enhance stratification and differentiation of the epithelium.

A further object of the invention relates to a human skin substitute obtainable by the method as above described.

A further object of the invention relates to a method for grafting an animal, preferably a mammal, more preferably a mouse, with a human skin substitute as described above. In a particular embodiment said animal is an immunodeficient animal (e.g. NOD/SCID mouse). Said method may be useful to provide animal models for human skin.

In a particular embodiment, animals grafted with a human skin substitute of the invention may be generated as described by Del Rio M. et al. (2002). Briefly, animals are shaved and aseptically cleansed. Full-thickness wounds are then created on the dorsum of mice and finally grafting with the human skin substitute of the invention is performed under sterile conditions. 10-12 weeks may be then sufficient to obtain a human skin on said animal.

A further object of the invention relates to an animal model for human skin obtainable according to the method as above described.

The human skin substitutes and animal models of the present invention may have a variety of uses. These uses include, but are not limited to, use for screening compounds, substrates for culturing tumors and pathological agents (e.g., human papilloma virus), and for modelling human injuries or pathologies associated with skin damage.

For example human skin substitutes and animal models of the present invention may be used for a variety of in vitro and in vivo tests. In particular but in non limiting way, the human skin substitutes and animal models of the present invention find use in the evaluation of: skin care products, drug metabolism, cellular responses to test compounds, wound healing, phototoxicity, dermal irritation, dermal inflammation, skin corrosivity, and cell damage. Typically, for animal models of the invention, the product may be administered topically on the human skin, or may be administered through an oral, sublingual, subcutaneous, intramuscular, intravenous, and transdermal route.

The present invention encompasses a variety of screening assays. In some embodiments, the screening method comprises providing a human skin substitute or an animal model of the present invention and at least one test compound or product (e.g., a skin care product such as a moisturizer, cosmetic, dye, or fragrance; the products can be in any from, including, but not limited to, creams, lotions, liquids and sprays), applying the product or test compound to said human skin substitute or animal model, and assaying the effect of the product or test compound on the human skin substitute or animal model. Typically, for animal models of the invention, the test compound or product may be administered topically on the human skin, or may be administered through an oral, sublingual, subcutaneous, intramuscular, intravenous, and transdermal route. A wide variety of assays may be used to determine the effect of the product or test compound on the human skin substitute or animal model. The assays may be directed to the toxicity, potency, or efficacy of the compound or product. Additionally, the effect of the compound or product on growth, barrier function, or tissue strength can be tested.

In other preferred embodiments, the human skin substitutes or animal models of the invention find use for screening the efficacy of drug introduction across the skin.

In a particular embodiment, the human skin substitutes or animal models of the present invention are also useful for the culture and study of tumours that occur naturally in the skin as well as for the culture and study of pathogens that affect the skin. Accordingly, in some embodiments, it is contemplated that the human skin substitutes or animal models of the present invention are seeded with malignant cells. These reconstructed human skin substitutes or animal models can then be used to screen compounds or other treatment strategies (e.g., radiation or tomotherapy) for efficacy against the tumour in its natural environment. In some embodiments of the present invention provide methods comprising providing a reconstructed human skin substitute or animal model infected with a pathogen of interest and at least one test compound or treatment and treating the skin substitute or animal model with the test compound or treatment.

In another particular embodiment, the human skin substitutes or animal models of the present invention are also useful for modelling human injuries or pathologies associated with skin damage. For example, the human skin substitutes and animal models of the present invention may provide both in vitro and in vivo models for modelling wounds, burns (e.g. fire burns, sunburns . . . ), or lesions caused by irradiations, pathogens . . . , irritations caused by chemical products or environment conditions, degenerative diseases and genetic diseases. In certain embodiments, pathologies of interest are genodermatosis such as Epidemolysis bullosa, Xeroderma pigmentosum, ichthyosis, ectodermal dysplasia, kindler syndrome and others. Typically, the human skin substitutes or animal models of the present invention may be generated form pluripotent stem cells that may carry a mutation or a plurality of mutations that are causative for a genetic disease of the human skin. Both in vitro and in vivo models as described above may have particular interests for medical research or may be useful for screening compounds for the treatment or the prevention of said injuries and pathologies. In particular, the present invention contemplates the use of the human skin substitutes and animal models according to the invention for screening of compounds from libraries, in particular combinatorial libraries, using e.g. high throughput or high content techniques. Typically, for animal models of the invention, the test compound or product may be administered topically on the human skin, or may be administered through an oral, sublingual, subcutaneous, intramuscular, intravenous, and transdermal route.

In a further aspect of the invention, the human skin substitutes of the present invention may be used for the treatment of a pathology associated with skin damage.

Therefore the present invention relates to a method for the treatment of a pathology associated skin damage comprising a step consisting of grafting a patient in need thereof with a human skin substitute of the invention.

For example, the human skin substitutes of the present invention find use in wound closure and burn treatment applications. The use of grafts for the treatment of burns and wound closure is described U.S. Pat. Nos. 5,693,332; 5,658,331; and 6,039,760. Accordingly, the present invention provides methods for wound closure, including wounds caused by burns, comprising providing a human skin substitute according to the present invention and a patient suffering from a wound and grafting the patient with the human skin substitute under conditions such that the wound is closed.

The invention will be further illustrated by the following figures and examples. However, these examples and figures should not be interpreted in any way as limiting the scope of the present invention.

### FIGURES

FIG. 1: Establishment of a keratinocyte lineage: Morphology microscopy analysis of hES cells at different steps of differentiation (0-10-25-40 days). Initially, typical hES cells colonies are round. At 10 days, derived hES cells from the periphery of the colonies started to migrate and to spread into the feeder layer. From the twenty days onwards, these cells increased in volume, flattened and acquired epithelial morphology. At the end of differentiation, these cells became to have the pavimentous epithelial morphology, formed colonies of tightly packed, cohesive cells, characteristic of keratinocyte morphology.

FIG. 2: Establishment of a keratinocyte lineage: Quantitative PCR analysis of cells derived from hES cells during the 40 days of differentiation. The pluripotency gene markers OCT4 and NANOG, decreased rapidly from 5 days to finally be undetectable at 20 days. The transcript of keratin 18 and keratin 8 (KRT18 and KRT8), first specific markers of simple epithelia, were strongly increased up to 10 days to next decreased and be stabilized at a basal level until the end of the differentiation. Transcripts encoding keratin 5 and keratin 14 (KRT5 and KRT14), specific of the proliferative basal layer of the epidermis, increased steadily between 10 and 40 days

FIG. 3: Establishment of a keratinocyte lineage: FACS analyses of hES cells during the 40 days of differentiation confirmed a loss of the marker of undifferentiatied state SSEA3 (Stage-Specific Embryonic Antigen) from near 60% at the beginning of the kinetic to near 1% at 40 days. Commitment to the epidermal lineage was observed at 10 days when the pick of expression of keratin 18 (K18) rise to 63%. After 25 days K18 decreased continuously until to reach a basal level at 40 day (9%). From 25 days, a switch between the marker of simple epithelia K8/K18 and K5/K14 markers of stratified epithelia occurred with 59% of derived hES cells positive for K14, confirming an enrichment of the culture in basal proliferative epidermal cells.

FIG. 4: Characterization of a homogenous and pure population of keratinocytes derived from hES cells: Morphology microscopy analysis of human primary adult keratinocytes (HK) and keratinocytes derived from hES cells (K-hES cells) after subsequent culture. After 40 days of differentiation, subsequent cultivation of keratinocytes derived from hES cells was done without BMP4 and ascorbic acid in FAD medium seeded onto mitomycin treated 3T3 feeder cells. Under these conditions, keratinocytes derived from hES cells (K-hES cells) presented the same colony morphology than the adult primary human keratinocytes (HK). K-hES cells formed colonies of tightly packed, cohesive cells, characteristic of keratinocyte morphology.

FIG. 5: Characterization of a homogenous and pure population of keratinocytes derived from hES cells: FACS analysis of HK and K-hES cells revealed a loss of K18 and a homogenous cell population of K-hES cells in which more than 95% of the cells expressed K5 and K14.

FIG. 6: Characterization of a homogenous and pure population of keratinocytes derived from hES cells: Quantitative PCR analyses of K-hES cells and HK with OCT4/NANOG, KRT8/KRT18, KRT5/KRT14, integrins alpha6 and beta4 (ITGA6/ITGB4), laminin-5 and collagen VII (LAMB3/Col7A1) gene markers of keratinocytes adhesion were performed. Gene expression levels were similar for all these tested genes that are characteristic of basal keratinocytes.

FIG. 7: Establishment of functional keratinocytes derived from hES cells. Colony forming assay of HK and K-hES cells. The growth potential of human keratinocytes in vitro can be estimated by the number of the growing adherent clones. Colony forming analysis of K-hES cells showed an increased of 40% of clonogenic potential of these cells compared to HK.

FIG. 8: Establishment of functional keratinocytes derived from hES cells. Organotypic epithelia culture of HK and K-hES cells. Hematoxylin/Eosin histological staining after 10 days of air liquid differentiation. The epidermal architecture appeared to be composed of a well-defined basal layer with a pavimentous cell shape and suprabasal layers, including a stratum granulosum containing keratohyalin granule and a stratum corneum, seen as superposed layers of dead squame enucleated cells.

FIG. 9: PCR Array on organotypic HK and K-hESC epidermis. A large panel of epidermis genes has been tested on cDNA extracted from HK and K-hES cells derived organotypic epidermis. Data were collected using home made keratinocyte-focused primer quantitative PCR arrays and heat map analysis performed on Array Assist software. The two organotypic epidermis presented very similar patterns of expression.

FIG. 12: Long-term in vivo human epidermal regeneration following xenografting to immunodeficient mice. a. Haematoxylin-eosin staining of artificial skin implants grafted with K-hESC. Scale bar is 50 μm. b. Immunoperoxydase staining using mAb SY-5 directed against human involucrin on artificial skin implants grafted with K-hESC is appropriately located in spinous and granular layers. Note that dermal background could be observed, due to the anti-mouse secondary antibody. Scale bar is 50 μm.

FIG. 13: Long-term in vivo human epidermal regeneration following xenografting to 4 immunodeficient mice. Haematoxylin-eosin staining of artificial skin implants grafted with K-hESC. Scale bar is 100 μm.

FIG. 10: Homogenous profile of K-hES cells in semi-defined serum-free medium: Quantitative PCR analysis of K-hES cells maintained in semi-defined serum-free medium, and in FAD medium with feeder cells demonstrated similar expression of the transcripts of keratin 5 and 14 (KRT5 and KRT14).

FIG. 11: Stable phenotype of K-hESC up to nine passages: Quantitative PCR analyses of K-hES cells at successive passages, up to 9, showed stable expression of genes associated to the keratinocyte phenotype, including KRT5, KRT14, ITGA6 and ITGB4.

FIG. 14: Establishment of a keratinocyte lineage: Quantitative PCR analysis: PCR analyses of K-hES cells and HK showed different expression levels of the K19, K3 and K12 genes.

FIG. 15: Expression of MHC class I (HLA-ABC) and class II (HLA-DR) proteins in hESC, K-hESC and HK

Representative FACS analysis of MHC class I (HLA-ABC) and class II (HLA-DR) expression in hESC, HK and K-hESC derived from H9.

FIG. 16: Establishment of a keratinocyte lineage using induced pluripotent stem cells

(A) Morphology analysis of induced pluripotent stem cells (iPS) and derived iPS at 40 days of differentiation

(B) Quantitative PCR analysis of OCT4/NANOG, KRT8/KRT18, KRT5/KRT14 of derived induced pluripotent stem cells (iPS) during the 40 days of differentiation.

FIG. 17: Characterization of keratinocytes derived from iPS.

(A) Microscopy analysis of keratinocytes derived from iPS (K-iPS) and from hESC (K-hESC), and human primary keratinocytes (HK) after subsequent culture.

(B Quantitative PCR analysis of OCT4/NANOG, KRT8/KRT18, KRT5/KRT14 and P63 in K-iPS, K-hESC and HK.

(C) Immunofluorescence analysis of keratins 5 and 14 in K-iPS, K-hESC and HK.

### EXAMPLE 1

### Method for Preparing a Population of Keratinocytes and a human skin substitute from hES

Material & Methods

Maintenance Culture of hES Cells.

The hESC(SA-01 and H9) were grown on a feeder layer of mouse fibroblast cells, STO (inactivated with 10 mg/ml mitomycin C and seeded at 30000/cm²) in DMEM/F12 (Sigma) supplemented with 20% (vol/vol) Knockout Serum Replacement (KSR, Invitrogen), 1 mM glutamine, 0.1 mM nonessential amino acids (Invitrogen), 4 ng/ml recombinant human bFGF (PeProTech) and 0.1 mM 2-mercaptoethanol at 37° C. under 5% CO2. For passaging, hESC colonies were cut and passages were done every 5 days.

Derivation of hES Cells in Keratinocytes.

For derivation, clumps were seeded onto mitomycin C-treated 3T3 fibroblasts in FAD medium composed of ⅔ DMEM, ⅓ HAM:F12 and 10% of fetal calf serum (FCII, Hyclone) supplemented with 5 μg/ml insulin, 0.5 μg/ml hydrocortisone, 10⁻¹⁰ M cholera toxin, 1.37 ng/ml T3, 24 μg/ml adenine and 10 ng/ml recombinant human EGF. The induction of ectodermal differentiation was done when 0.5 nM of human recombinant BMP-4 (R&D Systems Europe, UK) and 0.3 mM ascorbic acid (Sigma) were added. Cells were grown in the same medium until clones of epithelial cells were isolated. Cells were then seeded in the same feeder layer in FAD medium devoid of BMP4 and ascorbic acid. As a control, primary human keratinocytes (HK) were cultured on mitomycin C treated 3T3 fibroblasts in FAD medium.

For culture in semi-defined serum-free medium, HK and k-hES cells were seeded on BioCoat Collagen I plastic (BD Biosciences) in KGM-2 medium (Lonza).

Quantitative PCR.

Total RNA was isolated from hES cells, HK and K-hES cells using RNeasy Mini extraction kit (Qiagen) according to the manufacturer's protocol. An on-column DNase I digestion was performed to avoid genomic DNA amplification. RNA level and quality were checked using the Nanodrop technology. A total of 500 ng of RNA was used for reverse transcription using the Superscript III reverse transcription kit (Invitrogen). To quantify mRNA expression real time RT-PCR analysis was performed using a LightCycler 480 system (Roche diagnostics) and SYBR Green PCR Master Mix (Roche Diagnostics) following the manufacturer's instructions. Quantification of gene expression was based on the DeltaCt Method and normalized on 18s expression. Melting curve and electrophoresis analysis were performed to control PCR products specificities and exclude non-specific amplification.

FACS Analyses

Cells were detached from culture plates using Trypsin-EDTA (Invitrogen) and fixed in 2% paraformaldehyde for 15 minutes at room temperature. After PBS wash, cells were permeabilized with 0.1% Saponin (Sigma). Primary antibodies diluted at 1:100 were incubated one hour at room temperature in PBS containing 0.1% FCS. Control samples were done using isotype specific or no primary antibody. Species specific secondary antibodies were added for 1 hour at room temperature and stained cells were analyzed on a FACScalibur flow cytometer using CellQuest software (BD Biosciences).

Immunocytochemistry.

Cells were fixed in 4% paraformaldehyde for 15 minutes at room temperature before permeabilized and blocking in PBS supplement with 0.4% Triton X-100 and 5% BSA (Sigma). Primary antibodies were incubated overnight at 4° C. in blocking buffer. Mouse anti-K14, rabbit anti-K14, mouse anti-K5 were purchased from Novacastra, mouse anti-ColVII, mouse anti-integrin α6 and mouse anti-laminin5 were from Santa-Cruz Biotechnology and mouse anti-integrin β4 was from BDbiosciences. Cells were stained with the species specific fluorophore-conjugated secondary antibody (Invitrogen) for 1 hour at room temperature and nucleus were dye using DAPI. Immunofluorescence images were acquired on a Zeiss Z1 microscope using Axiovision imaging software.

Colony Forming Assay.

Primary keratinocytes and K-hES cells were trypsinized and plated on mitomycin C treated 3T3 fibroblasts feeder layer in FAD medium at 14 cells/cm2 in a 10-cm plates. Cells were cultured for 2 weeks before being fixed with 70% ethanol and stained with Blue-RAL 555 (Sigma). After a tap water wash, plates were dried and colonies were counted. Each experiment was done in triplicate.

Organotypic Cultures.

Human skin substitute was generated as detailed elsewhere (Poumay Y et al., 2004). Keratinocytes cultures were performed on polycarbonate culture inserts (NUNC). These cells were maintained for 11 days in Epilife medium supplemented with 1.5 mM CaCl2 and 50 μg/ml ascorbic acid. The cells were exposed to the air-liquid interface by removing the culture medium for 10 days.

Grafting onto Immunodeficient Mice.

Bioengineered skin equivalents were generated using fibrin matrix populated with human fibroblasts. K-hESC were seeded on the fibrin matrix, grown immersed to confluence, and then, grafted on the back of 6-week-old female nu/nu mice (Jackson Laboratory, Bar Harbor, Me.) as described (Del Rio et al., 2002). Implants were harvested 10-12 weeks after grafting, and the tissue specimens fixed in 10% buffered formalin for paraffin embedding.

Array-Based Comparative Genomic Hybridization

Array-based comparative genomic hybridization (a-CGH) analysis was done using Integragen Chip genome-wide BAC array of 5245 BAC clones (526 kb median spacing).

Results:

hES cells (SA-01 or H9) were seeded on 3T3 feeder cells previously treated with mitomycin C in FAD medium supplemented with BMP4 (0.5 nM) and ascorbic acid (0.3 mM) and harvested at different time points 10, 25 and 40 days.

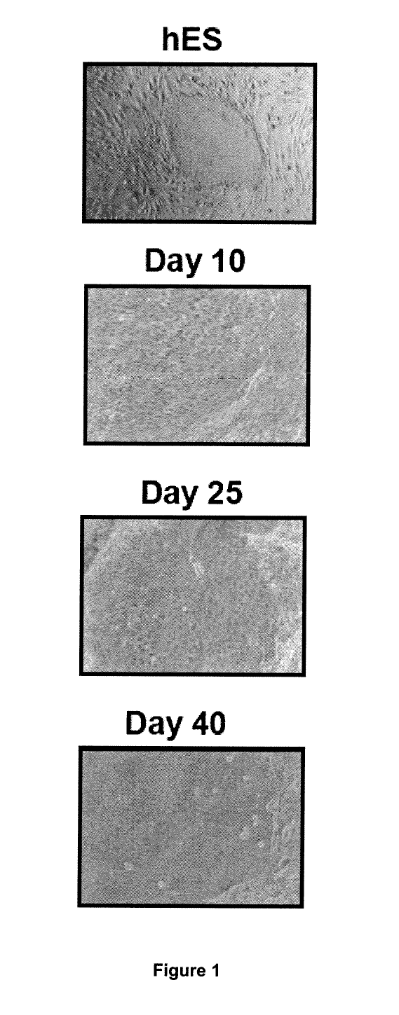

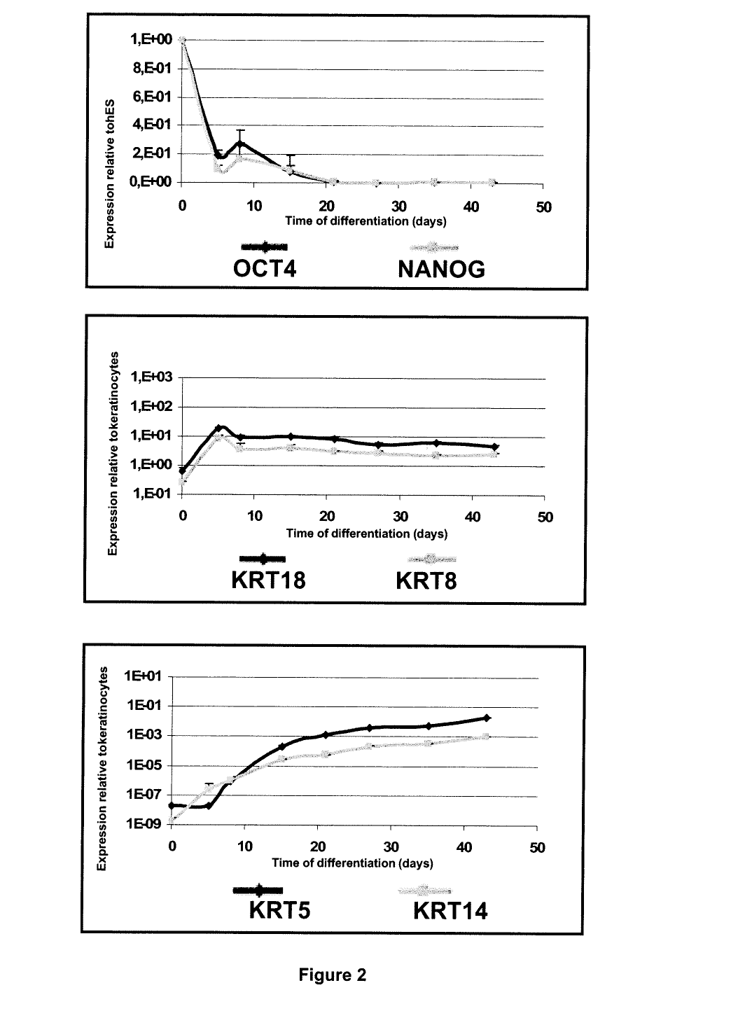

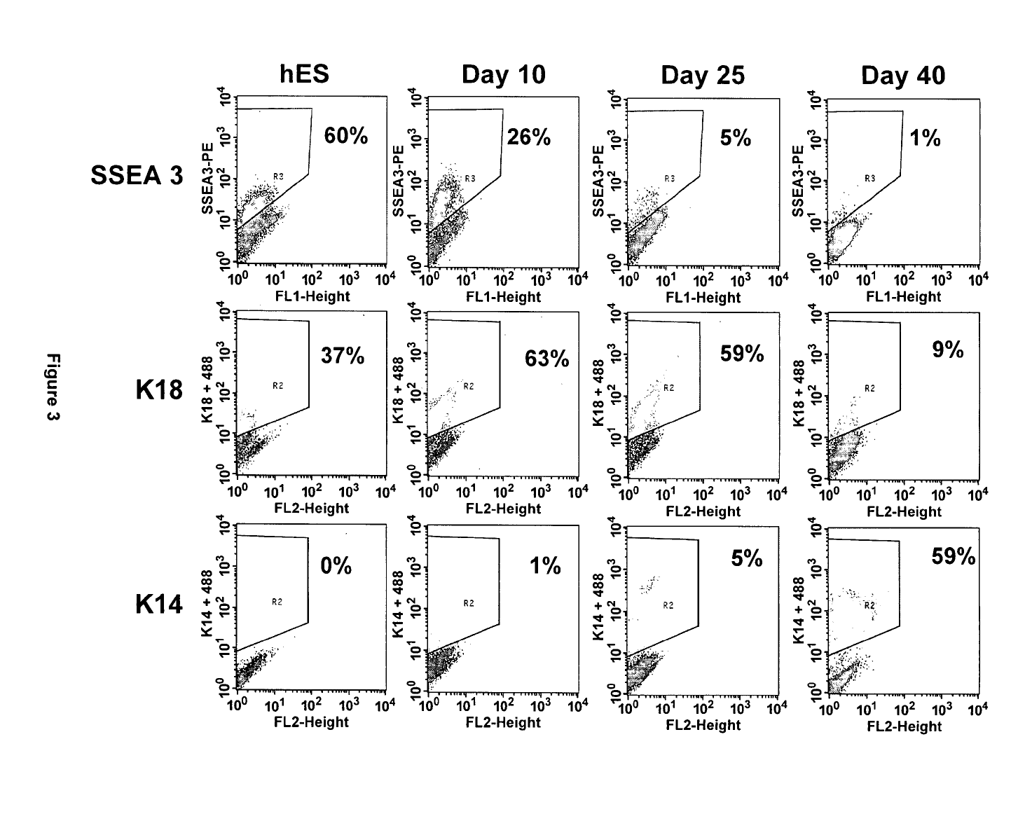

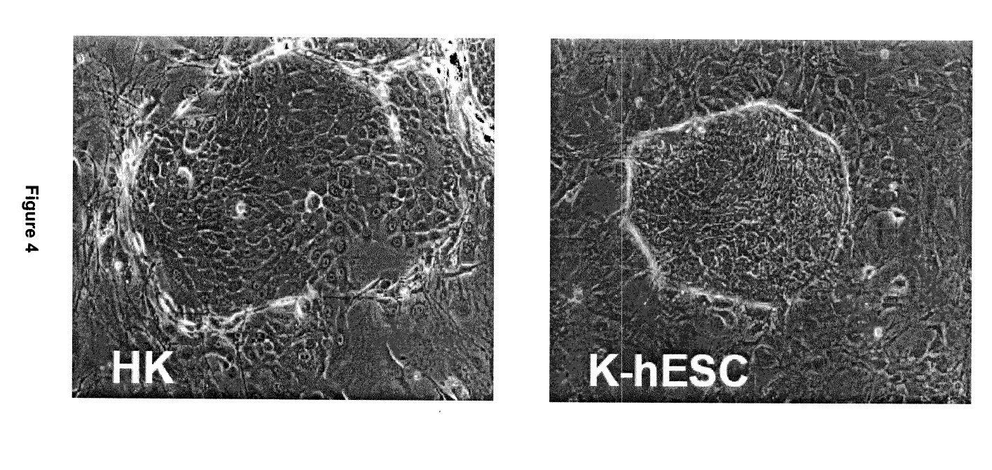

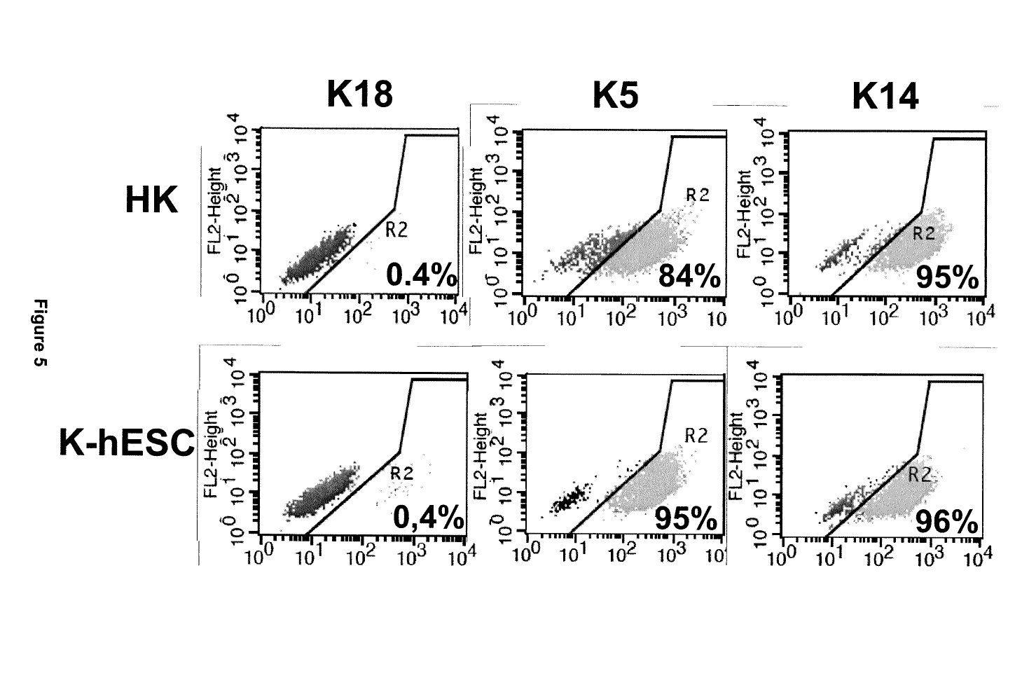

During the kinetic of differentiation, we observed by microscopy a gradually increased of epithelial morphology. Originally, undifferentiated hES cells formed single-cell layer colonies in culture. At 10 days, derived hES cells from the periphery of the colonies started to migrate and to spread into the feeder layer. From the twenty days onwards, these cells increased in volume, flattened and acquired epithelial morphology. At the end of differentiation, these cells became to have the pavimentous epithelial morphology, formed colonies of tightly packed, cohesive cells, characteristic of keratinocyte morphology (FIG. 1). A molecular characterization of the differentiation of hES cells was done all along the kinetic by quantitative-PCR and FACS analyses. Time course q-PCR analysis demonstrated that the pluripotency gene markers OCT4 and NANOG (Amit M. et al. 2000), decreased rapidly from 5 days to finally be undetectable at 20 days (FIG. 2). Specifically, FACS analysis confirmed a loss of the marker of undifferentiated state SSEA3 (Stage-Specific Embryonic Antigen) from near 60% at the beginning of the kinetic to near 1% at 40 days (FIG. 3).

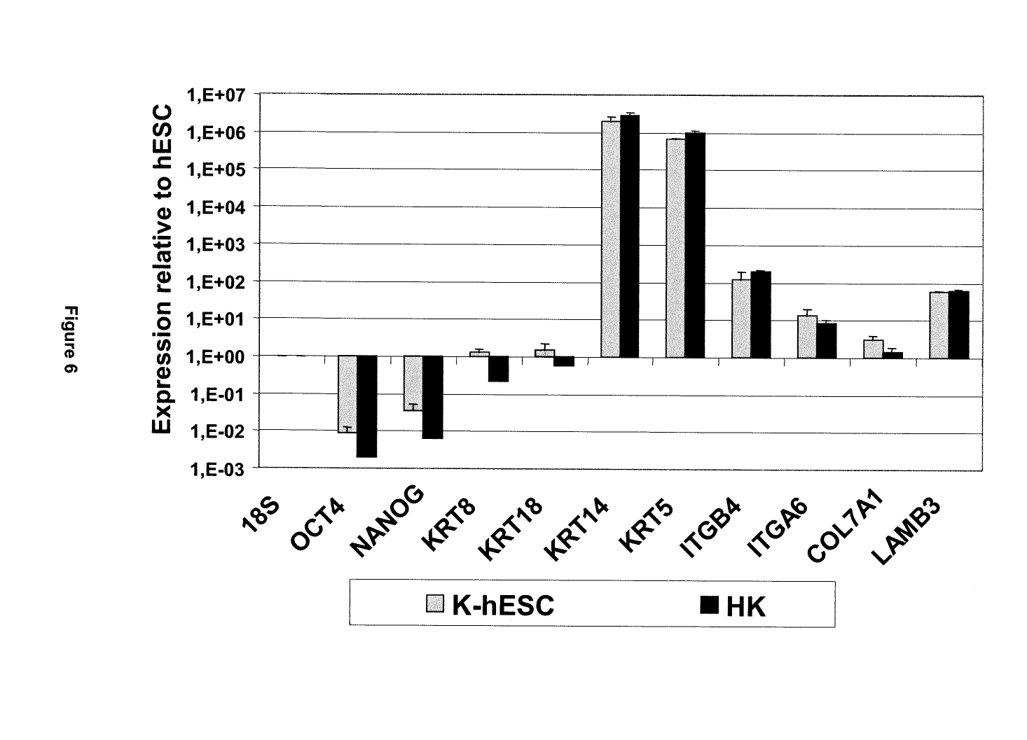

Epidermis development in vivo is characterised by temporal expression pattern of structural molecules during embryonic development (Mack J A. et al. 2005). The epidermis is derived from the ectoderm that gives rise to the single-layer ectodermal cells expressing the keratin 8 and the keratin 18 (K8 and K18). Using quantitative Q-PCR, expression of genes encoding the earlier markers along the keratinocyte lineage, keratin 18 and 8 (KRT8/KRT18) peaked at 10 days in culture then decreased progressively over the following weeks. Expression of genes encoding keratin 5 and 14 (KRT5/KRT14), which are specific of the proliferative basal layer of the epidermis all along life, increased progressively from day 10 on (FIG. 1B). FACS analyses confirmed the transitory expression of K18 with a pick of expression between 10 and 25 (63% to 59%) days consistent to a decreased until a basal level at 40 days (9%). At the end of the 40 days of differentiation we confirmed an enrichment of the culture in keratin 14 (59%) (FIG. 3). Finally, the derivation of hES cells to keratinocytes is comparable to epidermal development in vivo (Byrne C. et al. 1994). Altogether, data obtained clearly demonstrate that this protocol of differentiation reproduce in vitro all steps of epidermal development given the chance to a better understanding of the molecular events that are responsible for this drastic transition K8/K18 to K5/K14 At 40 days of differentiation. We considered that the period of induction was over, and stopped it by withdrawing BMP4 and ascorbic acid from the medium. After passage onto mitomycin C treated 3T3 feeder cells in FAD medium, cells exhibiting typical pavimentous epithelial morphology spontaneously formed growing colonies; we named them “keratinocytes derived from human embryonic stem cells” (K-hES cells). (FIG. 4). FACS analysis after four passages revealed no more keratin 18 and a quite homogenous cell population in which more than 95% of the cells expressed keratin 5 and 14 as in HK (FIG. 5). Comparison of K-hES cells with HK pointed to similar phenotypes. Gene expression levels as assessed by Q-PCR were similar for all tested genes that are characteristic of basal keratinocytes, including those encoding keratin 14, keratin 5, integrins alpha6 and beta4, collagen VII and laminin-5 (FIG. 6). Localization of keratin 5 and 14 were determined by immunofluorescence in cell compartments of K-hES cells identical to those in which they are observed in HK. As expected any staining for Oct4 and some remaining K18 staining were observed. Keratin 10, a marker of more differentiated keratinocytes of suprabasal layers, was absent, confirming the phenotypic characterization of K-hESC as basal keratinocytes. Adhesion capacity of these cells was suggested by the localization of integrins alpha6 and beta4 at the membrane, and that of laminin-5 and collagen VII in the extracellular matrix.

The characterization of K-h ES cells obtained in our condition shows that cells were closely identical to HK in culture. In addition derivation of hES cells offers an efficient means of generating a substantially pure homogenous population of keratinocytes with the same genetic background.

However, in addition to the typical markers of human adult primary keratinocytes, K-hES cells expressed significant levels of keratin 19 (a marker of skin stem cells in vivo and in vitro, which is expressed only in a few keratinocytes of the interfollicular epidermis and keratinocytes of the hair follicle) and of keratin 3 and 12 (corneal cell markers) (see FIG. 14).

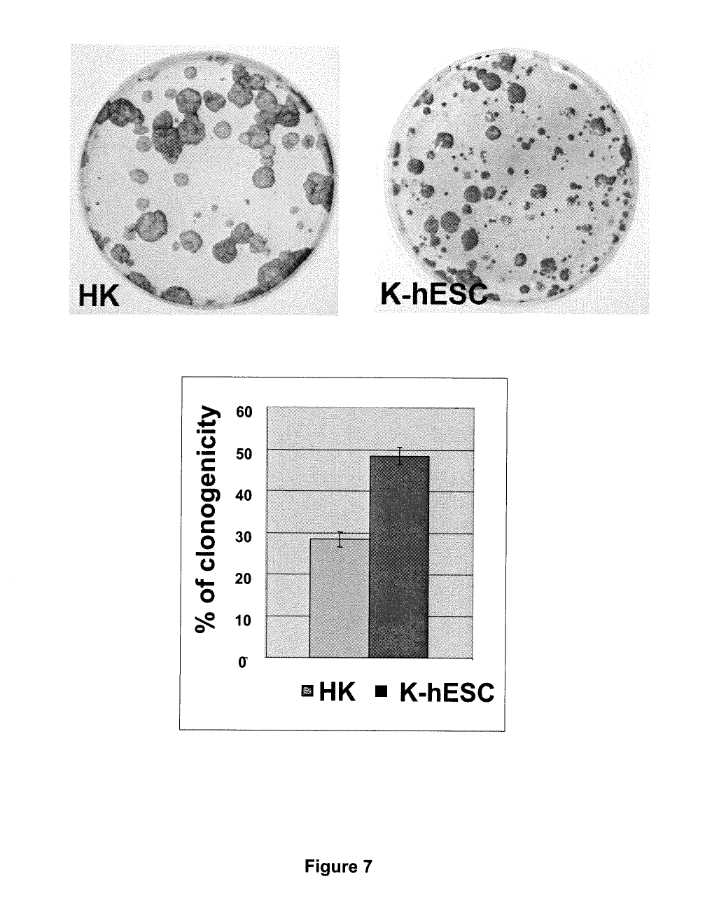

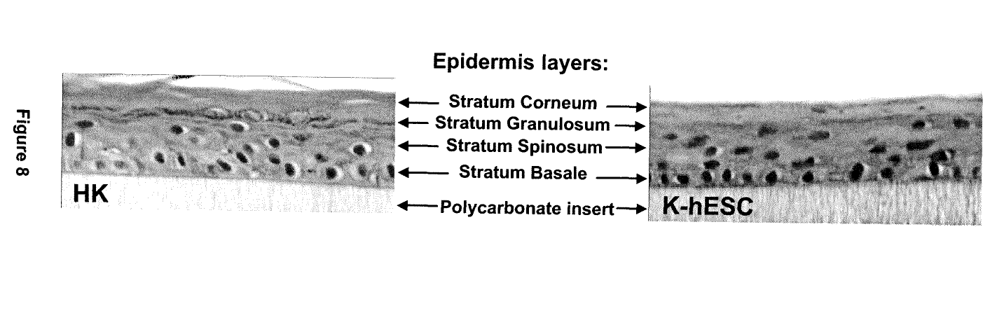

The generally accepted criteria used to defined keratinocytes in vitro are their capacity to form colony in cell culture systems. The growth potential of human keratinocytes in vitro can be estimated by the clone number that they are able to generate (Barrandon Y. et al. 1985). Interestingly, colony forming analysis of K-hES cells showed an increased of at least 40% of clonogenic potential (FIG. 7).

To test their physiological relevance, the K-hES cells were evaluated for their capacity to produce a pluristratified epidermis (FIG. 8). Reconstituted epidermis was generated in vitro using K-hES cells. After 10 days of air-liquid differentiation, histological staining of cryosection of organotypic cultures of K-hES cells showed the reconstitution of a stratified epidermis (Poumay Y. et al. 2004). The epidermal architecture appeared to be composed of a well-defined basal layer with a pavimentous cell shape and suprabasal layers, including a stratum granulosum containing keratohyalin granule and a stratum corneum, seen as superposed layers of dead squame enucleated cells. The normal morphological organization of the K-hES cells derived epidermis was also reflected in the regular expression and localization of differentiation markers, as analysed by indirect immunofluorescent staining. As expected, K14 staining was observed in the basal compartment of the reconstituted epidermis but was negative for the other suprabasal layers. K10 was present in the entire differentiated layer, just above the K14 positive single basal layer. Finally, fillagrin and involucrin, late markers of keratinocytes differentiation were detected exclusively in the most upper layers of the epidermis. Presences of late markers at the expected sites were indicators that our organotypic K-hES cells cultures had followed the physiological pathway toward differentiation.

To assess whether a basement membrane zone was found under the culture conditions used, the expression of adhesion molecules was examined in the reconstituted skin. The adhesion capacity of these cells was confirmed by a good localization of the integrin alpha 6 and beta 4 at the membrane of basal cells. In addition, the secretion of laminin-5 and collagen VII, extracellular matrix proteins allowing adhesion between the epidermis and the dermis were observed.

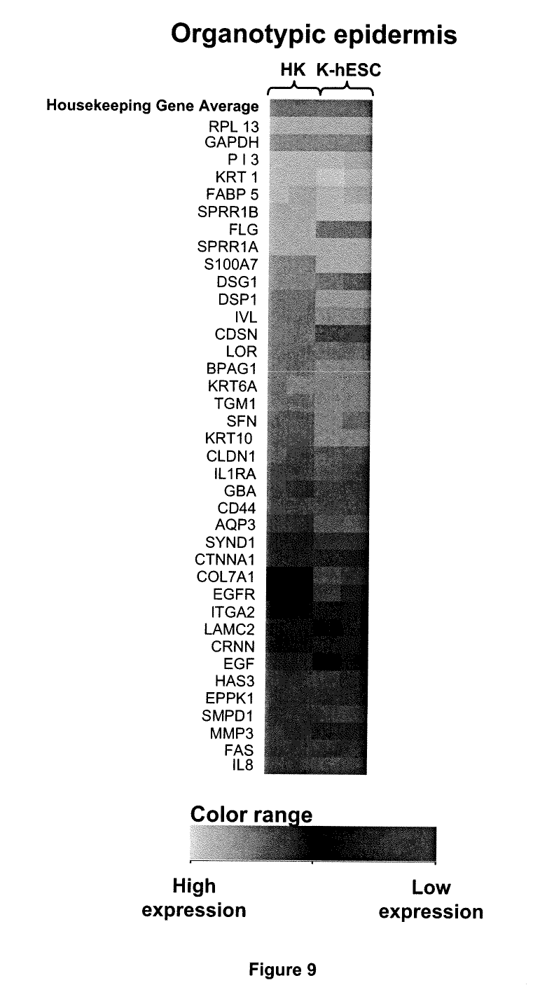

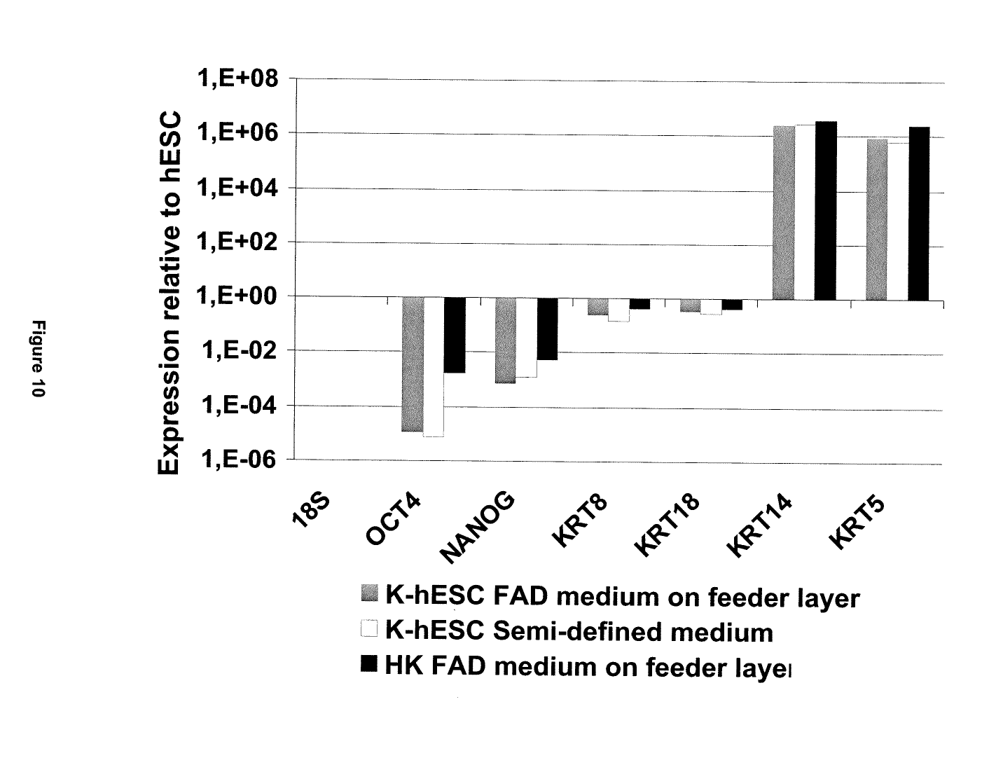

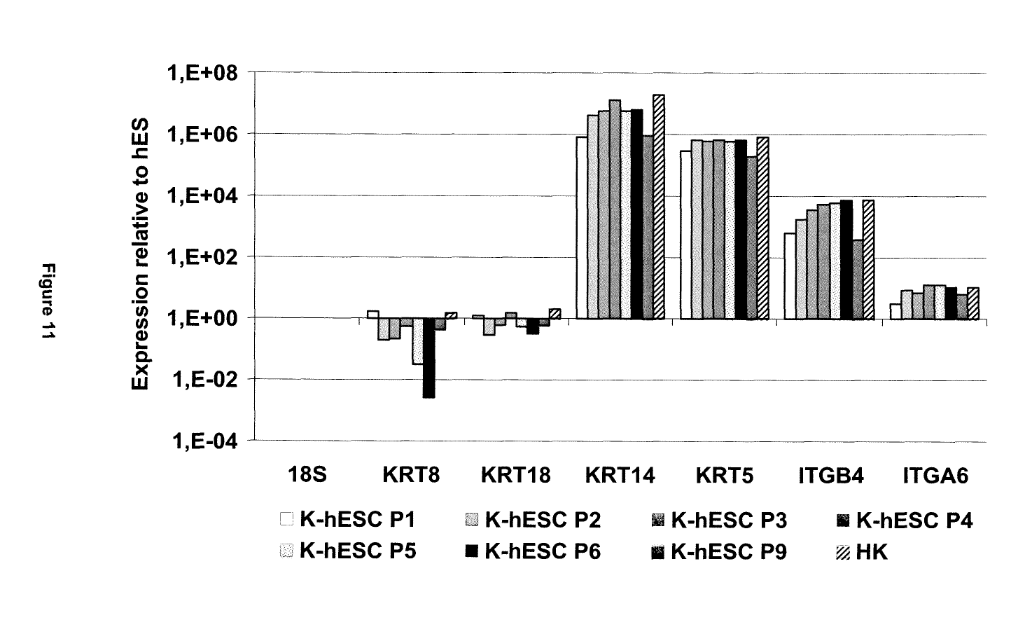

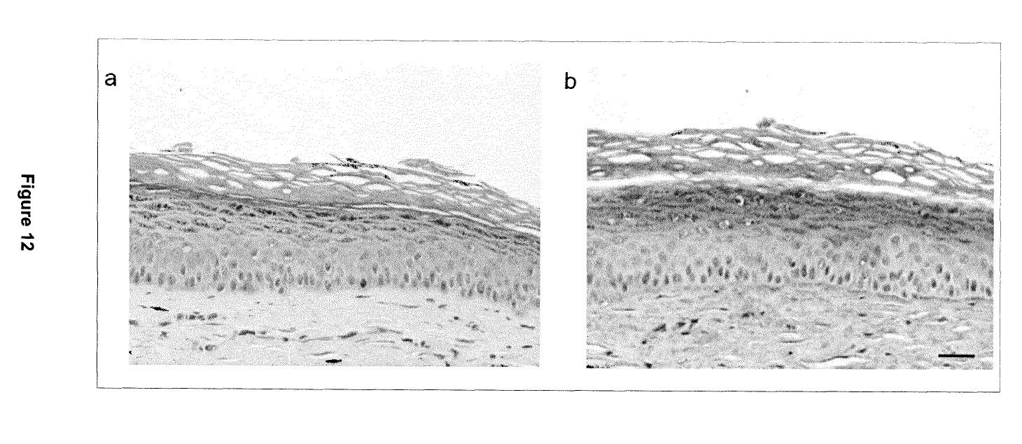

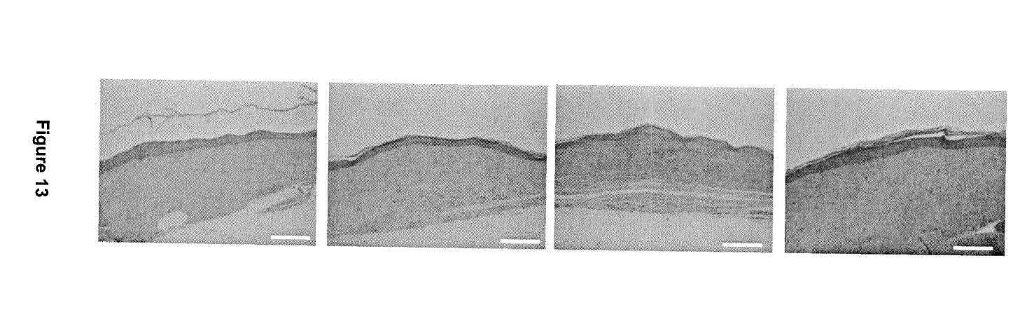

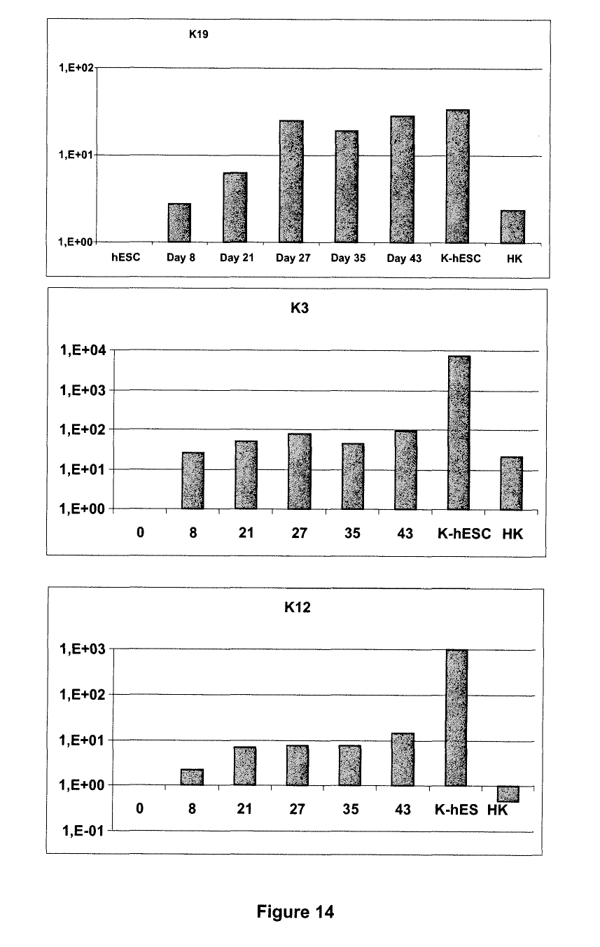

Moreover, a PCR Array using a panel of epidermis genes revealed that HK and K-h ES cells organotypic epidermis displayed very similar patterns of expression (FIG. 9).

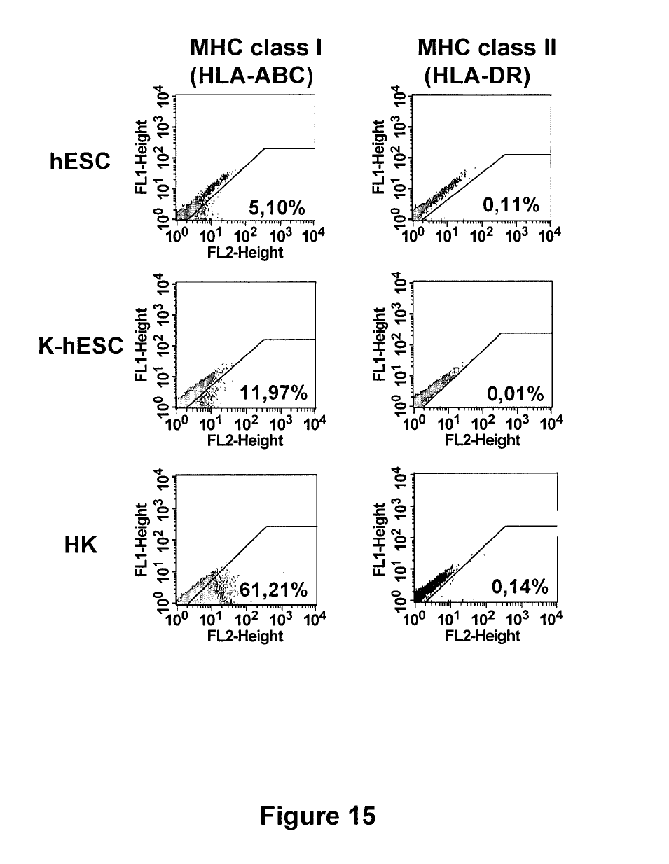

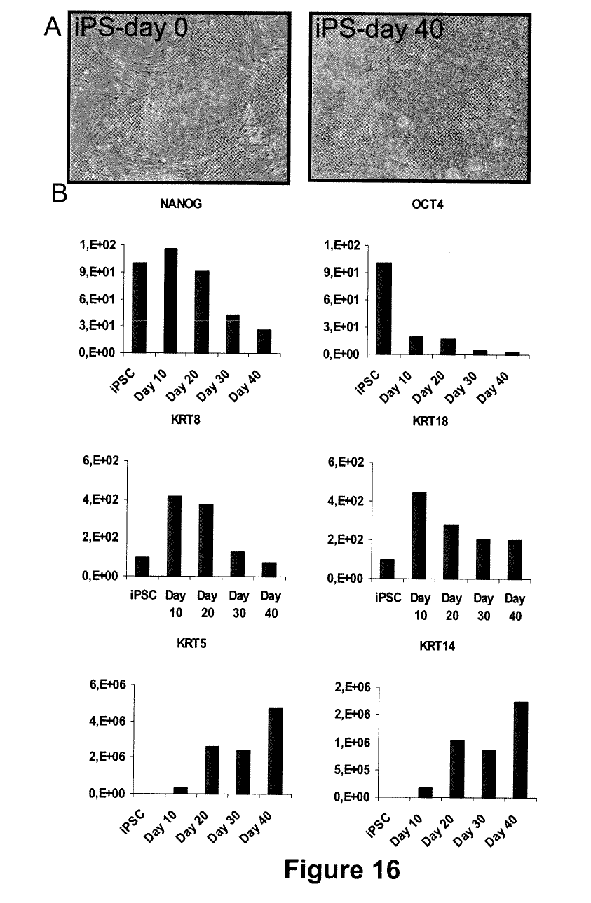

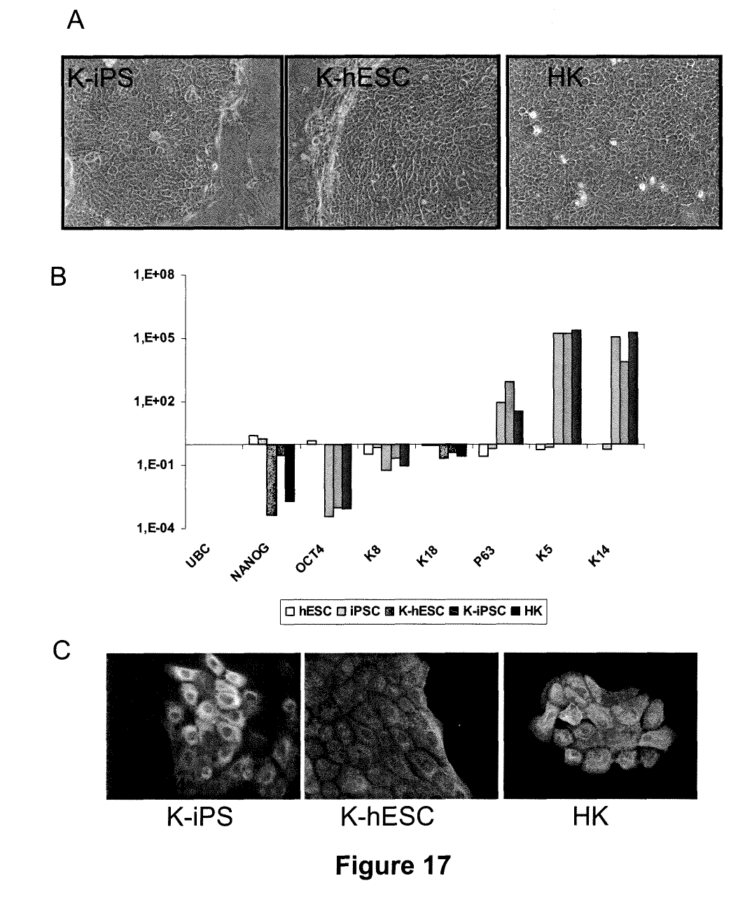

As a final demonstration, the capability of the K-hESC to generate self-renewing epithelia was evaluated through a stringent in vivo test. Fibrin matrix containing adult human fibroblasts were seeded with K-hESC to obtain confluent epidermal layer in vitro. These organotypic cultures were then grafted onto the dorsal region of immunodeficient nu/nu mice by orthotopical grafting (Del Rio M. et al. 2002; Larcher F. et al. 2007). 10-12 weeks later, K-hESC-derived epidermis from 4 mice on 5 exhibited a morphologically normal pluristratified architecture, consistent to that of mature native human skin (FIG. 12a and FIG. 13). Immunoreactivity for human involucrin was appropriately located in spinous and granular layers (dermal background due to secondary antibody) (FIG. 12b). This long-term in vivo regenerative features clearly indicate that K-hESC possess functional abilities of epidermal stem cells.

For clinical application, it's essential to use in vitro culture protocols devoid of animal or human products. The ideal culture medium for promoting the proliferation or terminal differentiation keratinocyte progenitor should be chemically defined and either be serum-free or synthetic serum replacement. Interestingly, we performed culture of K-hES cells in KGM2, a serum-free medium without feeder layers. Immunofluorescence analysis of K-hESC growing in KGM2 showed a homogenous expression of keratin 5, 14, and integrins alpha-6 and beta-4. Quantitative PCR analysis of K-hES cells maintained in semi-defined serum-free medium, and in FAD medium with feeder cells demonstrated similar expression of the transcripts of keratin 5 and 14 (FIG. 10).

The main result of the present study is the demonstration that cells derived from hESC are able to recapitulate all morphological and functional attributes of adult human keratinocytes in vitro and in vivo. This was obtained by treating undifferentiated ES cells using a protocol based on co-culture with cells that support ectodermal differentiation, preferably associated with long-term and low concentration BMP4 treatment able to stimulate epidermal induction and inhibit trophoblast and mesoderm induction. Ascorbic acid was added to stimulate terminal differentiation of keratinocytes in the absence of retinoic acid that was used by other authors (Bamberger C. et al. 2002). Successful outcome of our protocol may also arise from the fact that treatment was continuously applied up to full differentiation of keratinocytes, which required 40 days in culture. At that stage, the culture was enriched in cells that closely compared to adult epidermal inter-follicular keratinocytes. As these cells can be maintained for up to 9 passages (FIG. 11), frozen and thawed at will, they may represent a practical intermediate step for mass cell production of human keratinocytes, and pluristratified epidermis.

Immunogenicity of K-hESC was analyzed by FACS. Unlike adult basal keratinocytes, K-hESC revealed only very low levels of HLA-ABC antigens, and no HLA-DR (FIG. 15). K-hESC express little antigen if any of the major histocompatibility complex, demonstrating a low immunogenicity of the skin substitute.

### EXAMPLE 2

### Method for Preparing a Population of Keratinocytes and a human skin substitute from iPS

The same protocol of differentiation as described in Example 1 was performed with human induced pluripotent stem cells (iPS). Briefly, iPS were seeded onto mitomycin C-treated 3T3 fibroblasts in FAD medium composed of ⅔ DMEM, ⅓ HAM:F12 and 10% of fetal calf serum (FCII, Hyclone) supplemented with 5 μg/ml insulin, 0.5 μg/ml hydrocortisone, 10⁻¹⁰M cholera toxin, 1.37 ng/ml triodothyronin, 24 μg/ml adenine and 10 ng/ml recombinant human EGF. The induction of ectodermal differentiation was done when 0.5 nM of human recombinant BMP-4 (R&D Systems Europe, UK) and 0.3 mM ascorbic acid (Sigma) were added. Cells were grown in the same medium until clones of epithelial cells were isolated. Cells were then seeded in the same feeder layer in FAD medium devoid of BMP4 and ascorbic acid. As a control, primary human keratinocytes (HK) were cultured on mitomycin C treated 3T3 fibroblasts in FAD medium.

As shown in FIGS. 16 and 17, the inventors have shown that an isolated substantially pure homogenous population of human keratinocytes can also be derived from induced pluripotent stem cells (K-iPS).

### REFERENCES

Amit, M. et al. Clonally derived human embryonic stem cell lines maintain pluripotency and proliferative potential for prolonged periods of culture. Dev Biol 227, 271-278 (2000).
Amit M; Itskovitz-Eldor J; Derivation and spontaneous differentiation of human embryonic stem cells. J Anat 200:225-232 (2002).
Bamberger C, Pollet D, Schmale H. Retinoic acid inhibits downregulation of DeltaNp63alpha expression during terminal differentiation of human primary keratinocytes. J Invest Dermatol. 2002 January; 118(1):133-8.
Barrandon, Y. & Green, H. Cell size as a determinant of the clone-forming ability of human keratinocytes. Proc Natl Acad Sci USA 82, 5390-5394 (1985).
Byrne, C., Tainsky, M. & Fuchs, E. Programming gene expression in developing epidermis. Development 120, 2369-2383 (1994).
Coraux, C. et al. Reconstituted skin from murine embryonic stem cells. Curr Biol 13, 849-853 (2003).
Del Rio M, Larcher F, Serrano F, Meana A, Muñoz M, Garcia M, Muñoz E, Martin C, Bernad A, Jorcano J L. A preclinical model for the analysis of genetically modified human skin in vivo. Hum Gene Ther. 2002 May 20; 13(8):959-68.
Dicker, A. J. et al. Functional characterization of cultured cells derived from an intraepidermal carcinoma of the skin (IEC-1). Exp Cell Res 258, 352-360 (2000).
Doucet O, Garcia N, Zastrow L Skin culture model: a possible alternative to the use of excised human skin for assessing in vitro percutaneous absorption. Toxicol In Vitro 12:423-430 (1998)
Edmondson, S. R. et al. Insulin-like growth factor binding protein-3 (IGFBP-3) localizes to and modulates proliferative epidermal keratinocytes in vivo. Br J Dermatol 152, 225-230 (2005).
Gambaro, K., Aberdam, E., Virolle, T., Aberdam, D. & Rouleau, M. BMP-4 induces a Smad-dependent apoptotic cell death of mouse embryonic stem cell-derived neural precursors. Cell Death Differ 13, 1075-1087 (2006).
Gangatirkar, P., Paquet-Fifield, S., Li, A., Rossi, R. & Kaur, P. Establishment of 3D organotypic cultures using human neonatal epidermal cells. Nat Protoc 2, 178-186 (2007)
Gache Y, Baldeschi C, Del Rio M, Gagnoux-Palacios L, Larcher F, Lacour J P, Meneguzzi G. Construction of skin equivalents for gene therapy of recessive dystrophic epidermolysis bullosa; Hum Gene Ther; 15 (10): 921-33 (2004)
Green, H., Easley, K. & Iuchi, S. Marker succession during the development of keratinocytes from cultured human embryonic stem cells. Proc Natl Acad Sci USA 100, 15625-15630 (2003).
Green H, The birth of therapy with cultured cells. BioEssays 30:897-903, 2008.
Hovatta O, Mikkola M, Gertow K, Strömberg A M, Inzunza J, Hreinsson J, Rozell B, Blennow E, Andang M, Ahrlund-Richter L. A culture system using human foreskin fibroblasts as feeder cells allows production of human embryonic stem cells. Human Reprod. 2003 July; 18(7): 1404-9
Iuchi, S., Dabelsteen, S., Easley, K., Rheinwald, J. G. & Green, H. Immortalized keratinocyte lines derived from human embryonic stem cells. Proc Natl Acad Sci USA 103, 1792-1797 (2006).
Kariniemi, A. L., Lehto, V. P., Vartio, T. & Virtanen, I. Cytoskeleton and pericellular matrix organization of pure adult human keratinocytes cultured from suction-blister roof epidermis. J Cell Sci 58, 49-61 (1982).
Kaur, P. & Li, A. Adhesive properties of human basal epidermal cells: an analysis of keratinocyte stem cells, transit amplifying cells, and postmitotic differentiating cells. J Invest Dermatol 114, 413-420 (2000).
Kaur, P., Li, A., Redvers, R. & Bertoncello, I. Keratinocyte stem cell assays: an evolving science. J Investig Dermatol Symp Proc 9, 238-247 (2004).
Kim, S., Wong, P. & Coulombe, P. A. A keratin cytoskeletal protein regulates protein synthesis and epithelial cell growth. Nature 441, 362-365 (2006).
Larcher F, Dellambra E, Rico L, Bondanza S, Murillas R, Cattoglio C, Mavilio F, Jorcano J L, Zambruno G, Del Rio M. Long-term engraftment of single genetically modified human epidermal holoclones enables safety pre-assessment of cutaneous gene therapy. Mol. Ther. 2007 September; 15(9):1670-6. Epub 2007 Jun. 19.
Li, A. & Kaur, P. FACS enrichment of human keratinocyte stem cells. Methods Mol Biol 289, 87-96 (2005).
Li, A., Pouliot, N., Redvers, R. & Kaur, P. Extensive tissue-regenerative capacity of neonatal human keratinocyte stem cells and their progeny. J Clin Invest 113, 390-400 (2004).
Li, A., Simmons, P. J. & Kaur, P. Identification and isolation of candidate human keratinocyte stem cells based on cell surface phenotype. Proc Natl Acad Sci USA 95, 3902-3907 (1998).
Mack, J. A., Anand, S. & Maytin, E. V. Proliferation and cornification during development of the mammalian epidermis. Birth Defects Res C Embryo Today 75, 314-329 (2005).
Metallo, C. M., Ji, L., de Pablo, J. J. & Palecek, S. P. Retinoic acid and bone morphogenetic protein signaling synergize to efficiently direct epithelial differentiation of human embryonic stem cells. Stem Cells 26, 372-380 (2008).
Moreau, M., Leclerc C. The choice between epidermal and neural fate: a matter of calcium. Int. J. Dev. Biol. 48; 75-84 (2004).
Munoz-Sanjuan, I. & Brivanlou, A. H. Neural induction, the default model and embryonic stem cells. Nat Rev Neurosci 3, 271-280 (2002).
Poumay, Y. et al. A simple reconstructed human epidermis: preparation of the culture model and utilization in in vitro studies. Arch Dermatol Res 296, 203-211 (2004).
Redvers, R. P., Li, A. & Kaur, P. Side population in adult murine epidermis exhibits phenotypic and functional characteristics of keratinocyte stem cells. Proc Natl Acad Sci USA 103, 13168-13173 (2006).
Rheinwald, J. G. & Green, H. Formation of a keratinizing epithelium in culture by a cloned cell line derived from a teratoma. Cell 6, 317-330 (1975).
Rheinwald, J. G. & Green, H. Serial cultivation of strains of human epidermal keratinocytes: the formation of keratinizing colonies from single cells. Cell 6, 331-343 (1975).
Takahashi K, Tanabe K, Ohnuki M, Narita M, Ichisaka T, Tomoda K, Yamanaka S. Induction of pluripotent stem cells from adult human fibroblasts by defined factors. Cell. 2007 Nov. 30; 131(5):861-72.
Thomson J A; J Itskovitz-Eldor; S S Shapiro; M A Waknittz; J J Swiergiel; VS Marshall; J M Jones. Embryonic stem cell line derived from human blastocysts. “Science 282: (1998) 1145-1147
Uchida, Y., Behne, M., Quiec, D., Elias, P. M. & Holleran, W. M. Vitamin C stimulates sphingolipid production and markers of barrier formation in submerged human keratinocyte cultures. J Invest Dermatol 117, 1307-1313 (2001).
Webb, A., Li, A. & Kaur, P. Location and phenotype of human adult keratinocyte stem cells of the skin. Differentiation 72, 387-395 (2004).
Wilson, P. A. & Hemmati-Brivanlou, A. Induction of epidermis and inhibition of neural fate by Bmp-4. Nature 376, 331-333 (1995).
Yu J, Vodyanik M A, Smuga-Otto, K, et al., Induced Pluripotent Stem Cell Lines Derived from Human Somatic Cells. Science 318, 1917-1920 (2007)

1. An ex vivo method for obtaining a population of human keratinocytes derived from human pluripotent stem cells comprising a step of co-culturing human pluripotent stem cells with cells that support ectodermal differentiation in presence of an agent that stimulates epidermal induction and an agent that stimulates terminal differentiation of keratinocytes.

2. An ex vivo method for obtaining a population of human keratinocytes derived from human pluripotent stem cells, said method comprising a step of culturing human pluripotent stem cells on a cell culture surface coated with a layer of feeder fibroblasts in the presence of a keratinocyte culture medium supplemented with BMP-4 and ascorbic acid.

3. The ex vivo method according to claim 1 wherein said human pluripotent stem cells are embryonic stem cells (hES cells) or human induced pluripotent stem cells (human iPS cells).

4. The method according to claim 2 which comprises a further step of culturing the human keratinocytes derived from human pluripotent stem cells on a cell culture surface coated with a layer of dermis fibroblasts in the presence of a keratinocyte culture medium devoid of acid ascorbic and BMP-4.

5. An isolated substantially pure homogenous population of human keratinocytes derived from human pluripotent stem cells obtained by the method according to claim 4.

6. A pharmaceutical composition comprising a substantially pure homogenous population of human keratinocytes derived from human pluripotent stem cells of claim 5 and optionally a pharmaceutically acceptable carrier or excipient.

7. A method for the treatment of a pathology associated with skin damage in a subject in need thereof comprising administering to said subject the isolated substantially pure homogenous population of human keratinocytes derived from human pluripotent stem cells according to claim 5.

8. A method for preparing a human skin substitute comprising a step consisting of providing an organotypic culture of the substantially pure homogenous population of human keratinocytes derived from human pluripotent stem cells according to claim 5.

9. The method according to claim 8 wherein the substantially pure homogenous population of human keratinocytes according to claim 5 is previously seeded on a cell culture matrix populated with human dermis fibroblasts.

10. A human skin substitute obtained by the method according to claim 8.

11. A method for screening compounds comprising a step consisting of applying said compounds to the human skin substitute according to claim 10.

12. A method for culturing tumours or pathological agents comprising a step consisting of seeding the human skin substitute according to claim 10 with said tumours or pathological agents.

13. A method for treating a pathology associated with skin damage comprising a step consisting of grafting a patient in need thereof with the human skin substitute according to claim 10.

14-15. (canceled)

16. The method according to claim 13 wherein the pathology associated with skin damage is a burn.

17. The method according to claim 13 wherein the pathology associated with skin damage is a wound.
